(19) United States
(12) Patent Application Publication
(10) Pub. No.: US 2016/0271408 A1
(43) Pub. Date: Sep. 22, 2016

(54) COMPACT, PORTABLE DEFIBRILLATOR AND RELATED METHODS

(71) Applicant: CARDIOSPARK LLC, Tucson, AZ (US)

(72) Inventors: Carter Newton, Tucson, AZ (US); Norman P. Soloway, Tucson, AZ (US)

(21) Appl. No.: 15/074,990

(22) Filed: Mar. 18, 2016

Related U.S. Application Data
(60) Provisional application No. 62/134,716, filed on Mar. 18, 2015.
(60) Provisional application No. 62/262,737, filed on Dec. 3, 2015.

(51) Int. Cl. A61N 1/39; A61N 1/04

(52) U.S. Cl. CPC A61N 1/3968; A61N 1/046; A61N 1/0484; A61N 1/3925

(57) ABSTRACT

A portable defibrillator and related methods thereof are provided. The portable defibrillator has a housing having a first housing piece separable from a second housing piece, wherein each of the first and second housing pieces has an electrode, wherein the housing has a special volume of less than about 26 inches (66 cm³), a linear three dimension sum of less than 14 inches (35.5 cm). A wire is connected between the first and second housing pieces. A battery and a capacitor are positioned within at least one of the first and second housing pieces. A controller controls release of a voltage from the battery to at least one of the electrodes of the first and second housing.

### CROSS REFERENCE TO RELATED APPLICATION

This application claims priority from U.S. Provisional Application Ser. No. 62/134,716 filed Mar. 18, 2015, and U.S. Provisional Application Ser. No. 62/262,737, filed Dec. 3, 2015, the contents of which are incorporated herein by reference.

### FIELD OF THE DISCLOSURE

The present disclosure is generally related to defibrillators and more particularly to a method and system for a compact, portable defibrillator.

### BACKGROUND OF THE DISCLOSURE

Sudden Cardiac Arrest (SCA), which is also known as Sudden Cardiac Death (SCD), is a leading cause of death among adults over the age of 40 in the U.S., and throughout the world. In the U.S. alone, more than 300,000 men and women of all ages experience SCD Tragically, nearly nine out of 10 victims die. In relation to other common lethal conditions, the number of people who die each year from SCD is roughly equivalent to the number of people who die from Alzheimer' s disease, assault with firearms, breast cancer, cervical cancer, colorectal cancer, diabetes, HIV, house fires, motor vehicle accidents, prostate cancer and suicides combined. Accordingly, it is evident that SCA is a costly and disruptive medical affliction on our society.

A paramount of medicine is the fact that the steady circulation of blood is crucial to the proper functioning of the human body. The circulation of blood is governed by the heart, whose expansion and contraction is in turn controlled by a regular pattern of electrical impulses. When this pattern of electrical impulses becomes chaotic or overly rapid, SCA may take place. Tragically, the victim typically collapses and dies unless he or she receives proper medical attention. The most successful therapy for sudden cardiac arrest is prompt and appropriate defibrillation. A defibrillator uses electrical shocks to restore the proper functioning of the heart. A crucial component of the success or failure of defibrillation, however, is time. Ideally, a victim should be defibrillated immediately upon suffering a sudden cardiac arrest, as the victim's chances of survival dwindle rapidly for every minute without treatment. In fact, research has determined that the full survival of an out-of-hospital cardiac death is 5-7%, where full survival is understood as normal brain and heart function, and this statistic has not changed significantly in the past 15 years.

While defibrillators are commonplace within medical facilities, Automated External Defibrillators (AEDs) are increasingly being installed in public places. AEDs are typically located in emergency response vehicles, medical facilities, and many public buildings. Efforts have been made to improve the availability of AEDs, so that they are snore likely to be in the vicinity of sudden cardiac arrest victims. Also, advances in medical technology have reduced the cost and size of AEDs. Some modern AEDs approximate the size of a laptop computer or backpack. Even small devices may typically weigh 10 pounds or more. Accordingly, they are increasingly found in public facilities (e.g., airports, schools, gyms, etc.).

Currently available AEDs, while effective, are still less than ideal for most situations. For example, while AEDs are readily available in public settings and arc not complicated to use, it has been found that untrained bystanders typically cannot, or will not, utilize devices even when they are easily accessible. Even when one does attempt to utilize an AED in a public setting, it can be a challenge to actually locate an AED. Specifically, when a person suffers from SCA in an airport or public building in which multiple AEDs have been distributed, the victim's companion or a stranger would have to locate and run towards the nearest AED, pull the device off the wall, and return to the collapsed victim to render assistance. During that time, precious minutes may have passed. According to some estimates, the chance of surviving a sudden cardiac arrest is 90% if the victim is defibrillated within one minute, but that chance declines by 10% for every minute thereafter. A defibrillator design that reduces the time to defibrillation by even two to three minutes will save more lives.

Despite the increasing presence of AEDs in public places, a central problem with combating ACS still remains: approximately 80% of sudden cardiac arrests occur at a private home or residence, not at a public building. Until AEDs are readily available for use in generally private places, such as homes and in cars, their effectiveness will remain significantly limited. More specifically, until AEDs are designed to be conveniently and regularly carried by non-professional rescuers, and are designed for quick, uncomplicated use, the effectiveness of AEDs may remain limited.

Some companies within the industry have put forth AED devices which are intended to be used in non-public places, but these devices largely remain unsuccessful due to their complicated use and their cumbersome size. For example, companies such as Philips®, Zoll Medical Corporation, and Cardiac Science Corporation, among others, have AED devices which are portable and have a size which is smaller than conventional AEDs installed in medical facilities. These portable AEDs, for example, have a linear three-dimension sum (height, width, and thickness) of approximately 20 inches. The most prevalent size is an AED having a linear three dimension sum of about 24 inches to 27 inches (61×69 cm) with the smallest devices have a linear three dimension sum of about 16.7 inches (42.5 cm), a size which is still too large to carry the AED in one's pants pocket, a small purse, or in a glove box of a car. The spatial requirements of an adequately-sized battery and appropriate storage for electrode pads prevent the AED from being sized smaller. Beyond size, these devices utilize electrode pads which need to be affixed to the correct locations on the patient's body. While a medical professional is trained to use these AED devices and successfully position the pads on the patient's body, the use of pads further complicates the AED for an untrained, first-time user, especially in light of the urgent, stressful situation which necessitates using an AED in the first place.

Thus, there exists a need for a reliable AED with a compact size and uncomplicated use that can be widely distributed including for home use and for use by untrained, first-time users.

### SUMMARY OF THE DISCLOSURE

Embodiments of the present disclosure provide a system and method for a portable defibrillator. Briefly described, in architecture, one embodiment of the system, among others, can be implemented as follows. The portable defibrillator has a housing having a first housing piece separable from a second housing piece, wherein each of the first and second housing pieces has an electrode, wherein the housing has a special volume of less than about 26 cubic inches (66 cm³) and a linear three dimension sum of less than about 14 inches (35.5 cm). A wire is connected between the first and second housing pieces. A battery and a capacitor are positioned within at least one of the first and second housing pieces. A controller controls release of a voltage from the battery to at least one of the electrodes of the first and second housing.

In a preferred embodiment there is provided a portable defibrillator which includes two defibrillator paddles that are packaged together by a frangible seal. The paddles include electrodes having electrically conductive contact surfaces which in a preferred embodiment be coated with electrically conductive gel or comprise a plurality of points or apexes. The paddles, which a particularly preferred embodiment, are shaped as padded gloves or mittens, to make them appear particularly user friendly, are electrically and physically connected by a flexible bridge member which preferably includes indicia which may take the form of words and/or graphics to guide in the use of the AED.

In such embodiment, the system may be characterized y one or more of the following features
(a) where the controller activates upon separation of the first housing piece from the second housing piece;
(b) where the electrodes of each of the first and second housing pieces further comprises a plurality of cone-shaped electrodes;
(c) where the electrodes of each of the first and second housing pieces further comprises wire barb electrodes;
(d) where a GPS chip is included in the housing;
(e) where a wireless transceiver is included in the housing;
(f) where the controller initiates a wireless communication external of the housing using the wireless transceiver;
(g) where the controller further comprises rhythm acquisition and interpretation circuitry for sensing patient cardiac rhythm;
(h) where the controller further comprises a manual mode and an automated mode, wherein in the manual mode a user directs the controller to release the voltage, and in the automated mode, the controller initiates the voltage release automatically;
(i) where, in the automated mode, the controller initiates an audible shock sequence;
(j) where the housing is hermetically sealed;
(k) where the housing has a spatial volume of less than 26 cubic inches (66 cm³);
(l) where the controller includes a. module for sensing a victim's cardiac rhythm to determine if defibrillation is appropriate;
(m) where the controller includes a self-test module to monitor for battery strength, electrode moisture, etc., and generate an alert signal in the event the defibrillator is in danger of not properly functioning; and
(n) where the controller includes a memory and speakers for providing voice prompting for the proper use of the defibrillator.

The present disclosure can also be viewed as providing methods of using a portable defibrillator. In this regard, one embodiment of such a method, among others, can be broadly summarized by the following steps: providing a portable defibrillator with a housing having a first housing piece and a second housing piece with a wire therebetween, wherein each of the first and second housing pieces has an electrode, wherein a battery and a capacitor are positioned within at least one of the first and second housing pieces, and wherein the housing has a linear three dimension sum of less than about 14 inches (35.5 cm); and separating the first housing piece from the second housing piece to activate the portable defibrillator, wherein, after activation, the controller controls a release of a voltage from the battery to at least one of the electrodes of the first and second housing.

Other systems, methods, features, and advantages of the present disclosure will be or become apparent to one with skill in the art upon examination of the following drawings and detailed description, It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a perspective view illustration of a portable defibrillator, in accordance with the present disclaimer.

FIG. 2 is a perspective view illustration of the portable defibrillator of FIG. 1 with the first housing piece separated from the second housing piece, in accordance with the first exemplary embodiment of the present disclosure.

FIG. 3 is a diagrammatical illustration of an electrode of the portable defibrillator of FIGS. 1-2 in position on a patient, in accordance with the first exemplary embodiment of the present disclosure.

FIG. 4 is a diagrammatical illustration of the portable defibrillator of FIGS. 1-2 in position on a patient, in accordance with the first exemplary embodiment of the present disclosure.

FIG. 5 is a flowchart illustrating a method for using the portable defibrillator of FIGS. 1-2, in accordance with the first exemplary embodiment of the present disclosure.

FIG. 6 is a side elevational view of a defibrillator in a closed or packaged state in accordance with the present disclosure;

FIG. 7A illustrates a side elevational view and FIG. 7B a top plan view of a defibrillator open for use in accordance with the present disclosure;

FIG. 8 diagrammatically illustrates a block diagram illustrating various circuit components of defibrillator in accordance with the present disclosure;

FIG. 9 diagrammatically illustrates the use of a defibrillator in accordance with the present disclosure;

FIG. 10 is a flow chart illustrating a method of using a defibrillator in accordance with the present disclosure.

### DETAILED DESCRIPTION

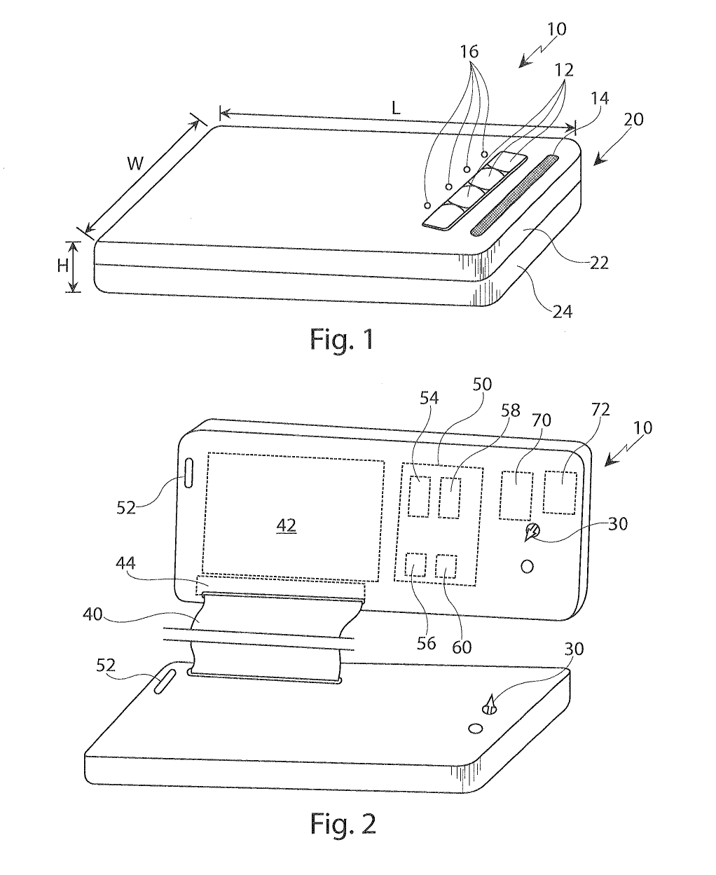

FIG. 1 is a perspective view illustration of a portable defibrillator 10, in accordance with the present disclosure. FIG. 2 is a perspective view illustration of the portable defibrillator 10 of FIG. 1 with the first housing piece separated from the second housing piece, in accordance with the first exemplary embodiment of the present disclosure. The portable defibrillator 10 is intended as a compact, user-friendly automatic external defibrillator (AFD), which can be conveniently carried in a pocket of a user's clothing, stored within a glove compartment of a vehicle, or maintained in a similarly small enclosure. Further, the portable defibrillator 10 has a simple design and is uncomplicated to use, which lessens the chances of incorrect and/or accidental uses of the portable defibrillator 10 and increases the likelihood of successfully combating sudden cardiac arrest (SCA) or sudden cardiac death (SCD). In comparison, conventional AEDs are large and conceptually complex, and using one in a public emergency situation can be intimidating, even potentially dangerous, for untrained users.

Relative to FIGS. 1-2, the portable defibrillator 10 has a housing 20 having a first housing piece 22 which is separable from a second housing piece 24. The housing 20 has a linear three dimension sum of less than about 14 inches (35.5 cm), where the linear three dimension sum is characterized as the summation of the length dimension (L), the width dimension (W), and the height dimension (H). This sizing of the housing 20 allows the portable defibrillator 10 to be small enough for convenient carrying and storage in a pocket of a user's clothing, within a glove compartment of a vehicle, or within other small enclosures, such as small purses, briefcases, and the like. In contrast to conventional defibrillator devices, a linear three dimension sum of less than about 14 inches is significantly smaller and easier to carry and transport, as conventional defibrillator devices are not sized to be carried within a pocket, a glove box, or similar compartment. As a point of comparison, the portable defibrillator 10 may have a size that is most similar to a plan size of a smart cellular phone, e.g., 5-6″ (12.7-15.3 cm) in length and 2.5-3″ (6.4-7.6 cm) in width, and may be slightly thicker than the ½″ size of a common smart cellular phone. Accordingly, in one design, the portable defibrillator 10 may be 6″ (15.3 cm) in length by 3″ (7.6 cm) in width, by 1 to 1.5″ (2.5-3.8 cm) in thickness. The size of the portable defibrillator 10 may also be understood in terms of other measurements, such as a spatial volume measure, which is characterized as the product of length, width and height. In this regard, the spatial volume of the portable defibrillator 10 may be less than 26 cubic inches (66 cm³).

The first and second housing pieces 22, 24 may connect together to form the housing 20 and they may he separated from one another when the portable defibrillator 10 is intended to be used. The engagement between the first and second housing pieces 22, 24 may be accomplished using various known designs and fasteners, such as biased fasteners which releasably engage between the first and second housing pieces 22, 24. Each of the first and second housing pieces 22, 24 has an electrode element or elements 30 carried on the structure. When connected together, the first and second housing pieces 22, 24 may be hermitically sealed and the electrodes 30 may be hermetically isolated from one another. The structure of the electrodes 30 may vary. In some examples, the structure of each of the first and second housing piece 22, 24 may be the electrode or electrodes 30 itself, e.g., where the electrode or electrodes 30 is a conductive surface of the first and second housing piece 22, 24. In other examples, the electrode or electrodes 30 may include a separate element which is incorporated into the first and second housing piece 22, 24. For example, unlike conventional gel-coated conductive pads which require direct contact with a patient's skin, the electrode or electrodes 30 may have a design which is capable of penetrating a patient's clothing, such as an electrode which is rigid, a cone-shaped, pointed, barb-shaped, a short stiff wire, or similar conductive elements 31. The electrodes 30 may also be incorporated into other designs to aid in utility of the device, such as by incorporating the electrodes 30 into a wearable belt.

The first and second housing pieces 22, 24 are connected with a wire 40, which is in electrical communication with each of the first and second housing pieces 22, 24. The wire 40 may also be used to physically connect the first and second housing pieces 22, 24 when the first housing piece 22 is separated from the second housing piece 24. The wire 40 may include a flat bridging wire, a single conducting wire, or any combination thereof. The wire 40 may be designed to efficiently store near or within the first and second housing pieces 22, 24 when the two structures are connected. For example, the wire 40 may have predetermined folds therein, or it may be retractable into the first or second housing 22, 24, or otherwise be organized within or proximate to the housing 20 when the portable defibrillator 10 is not in use.

At least one battery 42 and at least one capacitor 44 are positioned within at least one of the first and second housing pieces 22, 24, as illustrated in FIG. 2. The battery 42 may be a small battery which is capable of meeting both the spatial size requirements of the portable defibrillator 10, such that it can be embedded within one of the first and second housing pieces 22, 24, and the electrical requirements of operating the device. The capacitor 44 may include a capacitor bank which, together with the battery 42, provides enough voltage for at least three full defibrillations (250 Joules, biphasic waveform for 10 msecs each defibrillation shock). The battery 42 may be a non-rechargeable battery which has a 5 year shelf life.

A central processor controller 50 is included with the portable defibrillator 10 controlling the various functions of the device, including activation, the sensing functions, operation of a shock sequence, and communications, among others. Initially, when the portable defibrillator 10 is not in active use, the controller 50 may remain in a sleep state to preserve battery power. Upon separation of the first housing piece 22 from the second housing piece 24, the controller 50 may automatically activate the portable defibrillator 10 such that manual activation or turning on of the device is riot needed. Various sensors 52 may be incorporated into the first and second housing pieces 22, 24 to detect when the structures are separated from one another. In another design, the activation of the portable defibrillator 10 may not use the controller 50, but may still be automated when the first and second housing pieces 22, 24 are separated. For example, a mechanical insulator or similar device may be positioned to block electrical activation of the portable defibrillator 10 until the first and second housing pieces 22, 24 are separated, at which point, separation of the structures causes the mechanical insulator to be moved, thereby allowing electrical activation of the portable defibrillator 10. Activation of the portable defibrillator 10 may include activation of any combination of the functions and/or components thereof, including the controller 50, processor modules, communication, battery control modules, shock power source/capacitor bank module, and others.

The controller 50 may ultimately control release of the shock voltage from the battery 42 to the electrode or electrodes 30 in either or both of the first and second housing pieces 22, 24. Prior to voltage shock release, however, the portable defibrillator 10 may utilize various other components to preform various functions to aid in voltage shock release. Each of these various components, which may include sensing components, processing components, communication components, and user-guiding components of the device, may be incorporated as individual hardware components and/or modules within the controller 50.

A sensing module 54 of the portable defibrillator 10 may utilize one or more sensors which can perform various patient sensing functions. For example, the sensing module 54 may receive electrode information, and may record and inform an analytic circuit (rhythm acquisition and interpretation circuitry) with a cardiac rhythm/signal of the patient. When the electrodes 30 of the portable defibrillator 10 are connected to the patient, the sensing module 54 may recognize the presence or absence of a heartbeat. Then, the sensing module 54, utilizing sensors, may acquire the ECG and discriminate through a detection module (ANSI/AMI DF80: 2003 stipulations may be required). A detection of Pulseless Ventricular Tachycardia (VT) or Ventricular Fibrillation (VF) or no rhythm may be run and a shock sequence may be activated. If a heartbeat of more than 120 is detected, no shock sequence is activated. Alternative sensing methods may detect the presence of a heartbeat using oxygen sat pulsations, faint mechanical oscillations, and others. If no mechanical cardiac activity is detected, or if only a faint mechanical heart activity, e.g., less than 120 is detected, a shock sequence may be activated. The sensing module 54 may use a detection module (not shown) to interpret sensor input (normally signal comparison to a waveform database) and inform a processor module.

A processor module 58 may be included within the controller 50 to provide computerized processing of the functions. For example, the processor module 58 may allow the user of the portable defibrillator 10 to provide input functions, such as using various buttons 12, and allow for audible and/or visual output instructions using a speaker 14 and/or indicator lights 16, as depicted in FIG. 1. The processor module 58 may also interface with the detection module and the battery 42, such that instructions can be output to a capacitor bank 44 or defibrillator waveform/capacitor bank circuit. The processing module 58 may include the operating system of the portable defibrillator 10 and therefor have management tasks upgradable firmware stored therein.

The controller 50 may further include an analytic circuit 56 to process and determine a shockable or not shockable situation, a determination of which can be communicated to the processor module 58. A shock circuit 60 may also be included with the controller 50 to charge the capacitor bank 44, then initiate the voltage shock. The sequence may be automated by the portable defibrillator 10 using an automatic mode, where an audible shock sequencers provided to the user. In one of many alternatives, the portable defibrillator 10 may be used in manual mode, where a shock sequence is controlled by a user. Manual mode of the portable defibrillator 10 may provide prompts or instructions to the user, using audible and/or visual indications, which guide the user on whether to initiate a voltage shock. In this operation, the portable defibrillator 10 effectively has a manually guided mode, where the user is provided with appropriate guidance to make a determination on whether to initiate a voltage shock.

The portable defibrillator 10 may include other features, such as Global Positioning Satellite (GPS) functionality through the use of an embedded UPS chip 70. A wireless transceiver 72 also may be included for sending and receiving wireless signals and communications external of the portable defibrillator 10. The wireless transceiver 72 may utilize any communication protocol, including standard cellular telephone bandwidth, Bluetooth bandwidth, and/or ANT+ protocols, among others. The portable defibrillator 10 may connect to a smart phone or another device to permit additional functionality, such as social networking and/or alerting functionality. Specifically, pairing of the portable defibrillator 10 with another device may allow 911 alerts to be initiated, neighbors to be notified, mesh network notification of connected devices with proximity awareness, a GPS beacon broadcast, and any combination thereof To illustrate an example of using the portable defibrillator 10, when a shock sequence is initiated automatically, the device may place a phone call to 911 to provide the GPS coordinates of the location of the patient.

Other functionality of the portable defibrillator 10 may include an automated battery self-test and having printed instructions positioned in an easily visible location on a component thereof. In use, the portable defibrillator 10 may act as a personal safety device and may become as common as a smoke detector, seatbelt, or fire extinguisher. The portable defibrillator 10 may be used as part of a community security program or pharmacy outreach, where patients with high risk of SCA or SCD can have a portable defibrillator 10 with them at all times. In some designs, the portable defibrillator 10 may be capable of pairing with or to complimenting health “wearables”, such as bracelets, watches, or similar devices. In this use, when the ominous signs or symptoms that may precede sudden cardiac arrest are identified, the device can be self-applied to function as a safety precaution in transit to an Emergency Room.

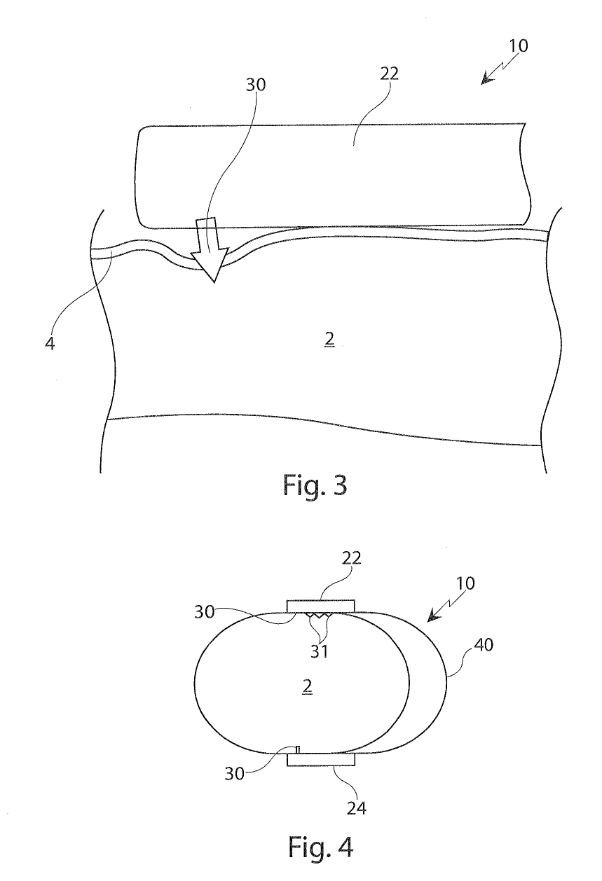

FIG. 3 is a diagrammatical illustration of an electrode 30 of the portable defibrillator 10 of FIGS. 1-2 in position on a patient, in accordance with the first exemplary embodiment of the present disclosure. FIG. 4 is a diagrammatical illustration of the portable defibrillator 10 of FIGS. 1-2 in position on a patient, in accordance with the first exemplary embodiment of the present disclosure. Relative to FIGS. 3-4, and incorporating the teachings of FIGS. 1-2, when the portable defibrillator 10 is used with a patient, the electrode or electrodes 30 may be capable of piercing the clothing of the patient. As shown in FIG. 3, the patient's body 2 is covered with an article of clothing 4, such as a shirt. When the first housing piece 22 of the portable defibrillator 10 is placed on the patient's body 2, the first housing piece 22 may be pressed towards the patient's body 2 such that the electrode or electrodes 30 can pierce the clothing 4 and make contact with the patient's body 2. While the electrode or electrodes 30 may cause a small abrasion on the patient's body 2, this side effect is significantly outweighed by the time-saving benefits of not having to remove the patient's clothing. FIG. 4 illustrates the portable defibrillator 10 in a position on a patient's body 2, where the first and second housing pieces 22, 24 connected together with the wire 40, and each having an electrode or electrodes 30, are positioned on opposing sides of the patient's body 2.

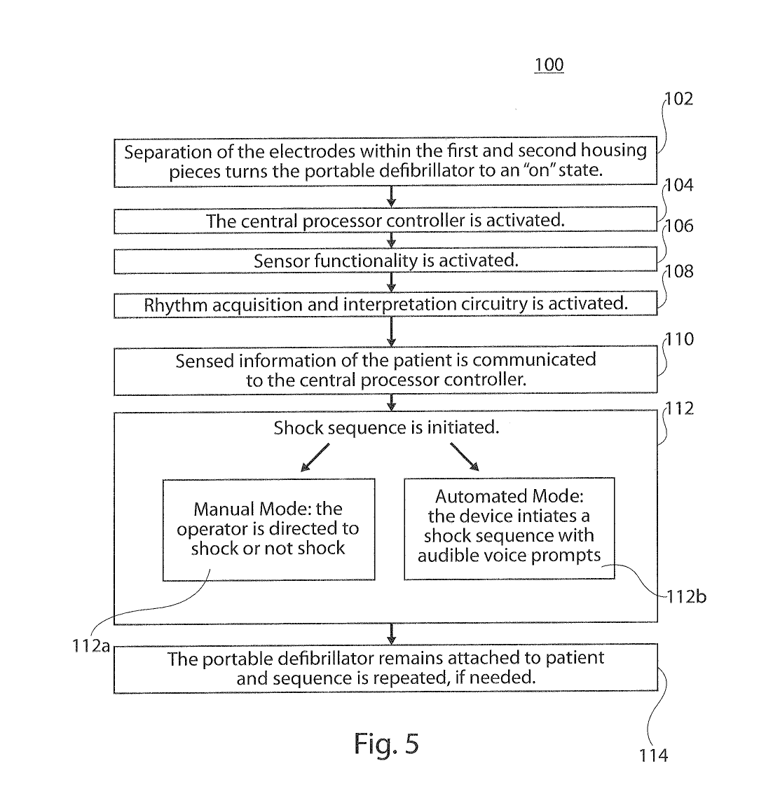

FIG. 5 is a flowchart 100 illustrating a method for using the portable defibrillator of FIGS. 1-2, in accordance with the first exemplary embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 102, separation of the electrodes within the first and second housing pieces turns the portable defibrillator to an “on” state. The central processor controller is activated (block 104). Sensor functionality is activated (block 106). Rhythm acquisition and interpretation circuitry is activated (block 108). Sensed information of the patient is communicated to the central processor controller (block 110). Shock sequence is initiated (block 112) ether manually where the operator is directed to shock or not shock (block 112a) or in an automated mode where the device initiates a shock sequence with audible voice prompts (block 112b). The portable defibrillator remains attached to patient and sequence is repeated, if needed (block 114).

In addition to the explicitly-noted steps identified in FIG. 5, a number of additional steps, processes, and functions may also be included. For example, during sensing, internal circuitry may record and analyze the victim's heart rhythm and determine if rhythm is “shockable” or “not shockable.” The internal circuitry may audibly warn and then deliver a defibrillation shock if appropriate, and may issue audible information that a defibrillation shock is not appropriate. The internal circuitry may sense dryness of gel electrodes, if used, or a low battery level, where either condition may activate a red warning LED on device. An algorithm for integrity self-check with LED status indicator may be used. One mode of the device may include a stand-alone, fully-autonomous functionality mode. A smartphone application may be activated to notify 911, to notify neighbors, to notify qualified first responders nearby, to direct additional control items to the device or broadcast audible instructions about frequency of CPR compressions. The device may pair with other wearable biometric devices and enable expanded device value to sense and to shock.

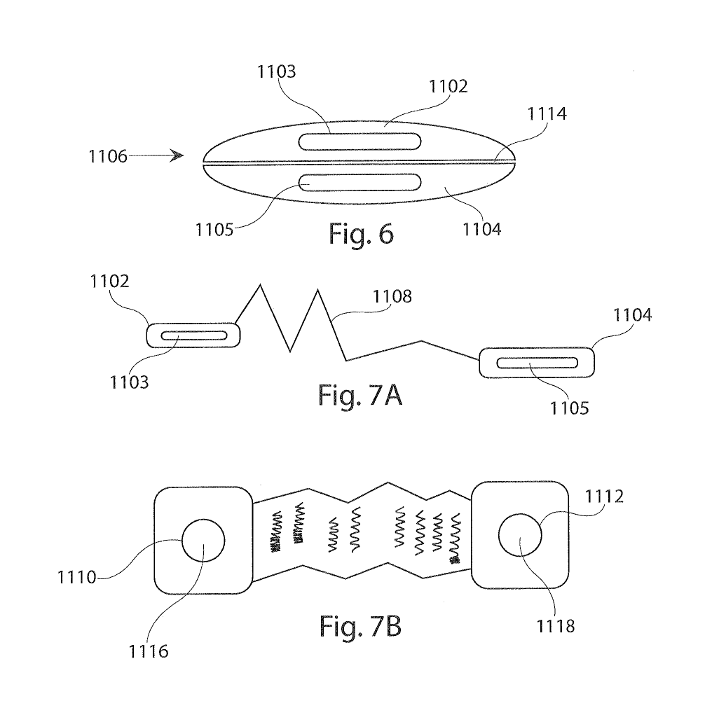

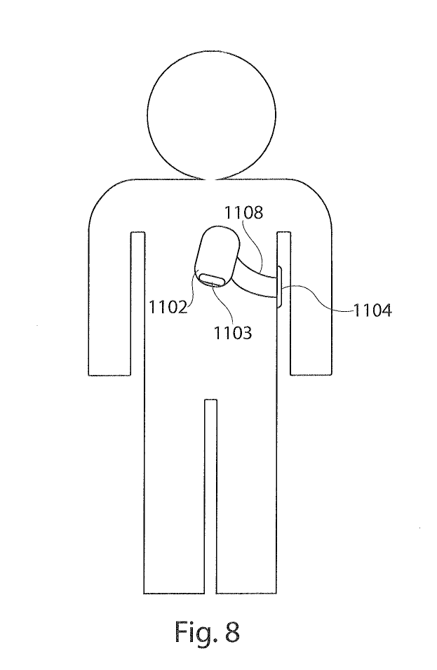

Referring now to FIGS. 6-8 of the drawings, a defibrillator 1100 in accordance with another embodiment of the present disclosure includes two defibrillator paddles 1102, 1104, which together fibrin a paddle module 1106. Paddles 1102, 1104 are electrically and physically connected to one another by a flexible bridge member 1108, and are packaged together with their electrode faces 1110, 1112, facing one another, and sealed as a module or unit by a frangible seal 1114. Paddles 1102, 1104 are sized and shaped as padded gloves or mittens, and include slots 1103, 1105 for a person's hands. Forming paddles 1102, 1104 as gloves or mittens provides significant advantages. Gloves or mittens are familiar and non-imposing. Thus, non-medical personnel may be less hesitant to use the defibrillator. This could save precious minutes in administration of defibrillation to a victim, and may be the difference between life and death. Electrode faces 1110, 1112 preferably are coated with electrically conductive surfaces 1116, 1118 which, in a preferred embodiment may comprise an electrically conductive gel.

Bridge member 1108 preferably includes indicia which may take the form of words and/or graphics to guide in the use of the defibrillator.

Included within one of the paddles 1102, 1104 or split between the paddles 1102, 1104 and/or the bridge 1108 is an electrical circuit module 120 which includes a capacitor 1122, control logic 1124 and battery 1126. Circuit module 120 preferably also includes a GPS module 1128 and an emergency calling device 1130 which preferably uses wireless communication to alert local personnel of an incident and location of the defibrillator 1100 when the frangible seal 1114 is broken. Thus, emergency personnel will be guided directly to the victim. Preferably, the hulk of the electronics including the battery are packaged within one, e.g., the left-hand paddle 1102. That way the other, e.g., right-hand paddle 1104 can be made much thinner which will permit the rescuer to slip the right-hand paddle under the victim's back (after rolling the victim on to his back). This would free up the right hand of the rescuer for CPR, to make phone calls, etc. Also, once the right-hand glove is positioned behind the victim's back, and the rescuer's hand slipped out, contact with the defibrillator electrode will be maintained by the weight of the victim pressing against the floor surface. Placing one electrode behind the back of the victim also provides an ideal shocking vector (back to front) with the left glove/electrode placed mid sternum.

In another and preferred embodiment of the disclosure, circuit 1120 also includes a module for sensing a victim's cardiac rhythm to determine that defibrillation is appropriate. Also, if desired, circuit 1120 may include a self-test module 1132 to monitor battery strength, electrode moisture, etc., and generate an alert signal in the event the defibrillator is in danger of not properly functioning. Also, if desired, circuit 1120 may include a memory device and speakers for providing voice prompts for proper use of the defibrillator.

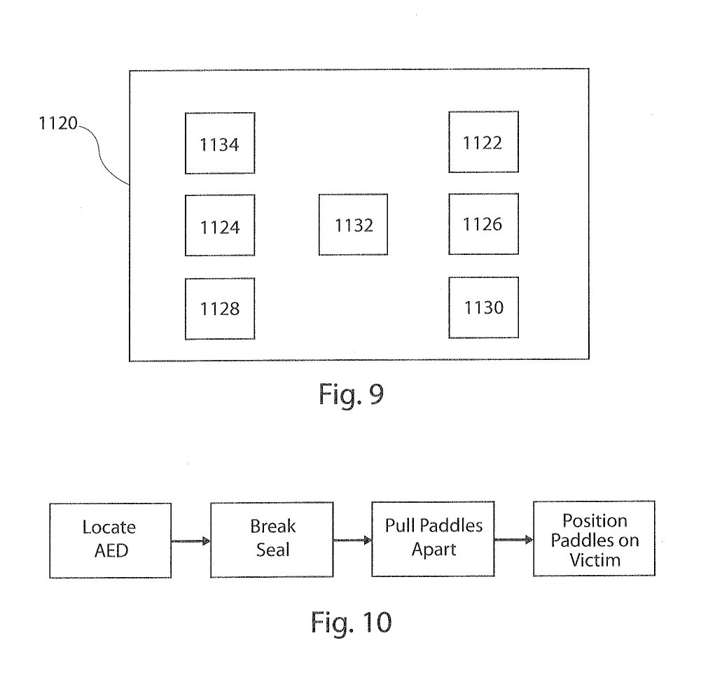

Referring also to FIGS. 9 and 10, there is shown use of a defibrillator in accordance with the present disclosure. Typically in the ease of sudden cardiac arrest, a rescuer locates the defibrillator, and breaks the frangible seal, pulling the paddles apart. On breaking the frangible seal, the defibrillator is activated, and immediately sends out an emergency call, e.g. to 911, or a private monitoring service such as a home security company, or a city emergency medical program. Activation of the defibrillator also sends a GPS location signal to assist emergency personnel in finding the location of the victim.

The rescuer places his hands in the glove-shaped paddles, breaks the frangible seal, spreads the paddles apart, and slides his right-gloved hand under the victim's back. The rescuer may then remove his right hand from the right-handed paddle. Preferably, but not necessarily, the right-handed paddle should be placed under the victim's clothing, in contact with the skin. However, it is not necessary to place the paddle in contact with the skin since the shock can penetrate clothing.

The defibrillator may be activated automatically, or the rescuer may push a button to activate the defibrillator. Once the defibrillator is activated, the rescuer should place his gloved left hand on the victim's sternum, again preferably in contact with the victim's skin. The defibrillator then checks the victim's ECG rhythm to determine whether it is shockable or not. If the defibrillator detects a shockable condition, the defibrillator charges its capacitor and discharges to an appropriate period.

Use of the portable defibrillator of the present disclosure may have significant benefits over conventional devices. For one, the portable defibrillator can be utilized as a fully FDA compliant personal defibrillator, since it is a smaller, easier-to-use, and more inexpensive home/automobile safety device than conventional defibrillators. Also, since the defibrillator is designed for limited, e.g. one-time use, and can be made quite low cost so that it may be widely distributed including in people's homes. It is simple to use and has many built-in fail safe features including battery-life monitor, electrode-pad monitor as well as emergency call and GPS monitoring. Furthermore, its design pennits a rescuer to use one hand, freeing the other hand of the rescuer for CPR, making phone calls, etc. And, since the portable defibrillator can be ubiquitously available due to its convenient carrying in nearby locations, such as pockets, purses, and glove compartments, the number of people who suffer complications and death due to SCA and SCD because a defibrillator is not available can be significantly reduced. Indeed, the expectation of patient survival in the home setting with the personal defibrillator can approach the well-documented hospital success rates of greater than 75%—7-fold improvement. Further, the design and size of the device can provide beneficial results within the industry since it is more intuitive and less threatening than conventional defibrillators.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A portable defibrillator comprising:
a housing having a first housing piece separable from a second housing piece, wherein each of the first and second housing pieces has at least one electrode, wherein the housing has a linear three dimension sum of less than 14 inches;
a wire connected between the first and second housing pieces;
a battery and a capacitor positioned within at least one of the first and second housing pieces; and
a controller controlling release of a voltage from the battery to at least one of the electrodes of the first and second housing pieces.

2. The portable defibrillator of claim 1, wherein the controller activates upon separation of the first housing piece from the second housing piece.

3. The portable defibrillator of claim 1, wherein the electrode of each or electrodes of the first and second housing pieces comprise cone-shaped or wire barb electrodes.

4. The portable defibrillator of claim 1, further comprising one or more features selected from the group consisting of a GPS chip, a wireless transceiver, wherein the controller preferably initiates a wireless communication external of the housing using the wireless transceiver, wherein the controller future comprises rhythm acquisition and interpretation circuitry for sensing patient cardiac rhythm, wherein the controller further comprises a manual mode and an automated mode, wherein in the manual mode a user directs the controller to release the voltage, and in the automated mode, the controller initiates the voltage release automatically, wherein in the automated mode, the controller preferably initiates an audible shock sequence, wherein the housing is hermetically sealed and wherein the housing has a spatial volume of less than about 26 cubic inches (66 cm³) and a linear three dimensional sum of less than about 14 inches (35.5 cm).

5. A portable defibrillator comprising:
two paddles electrically connected to one another, each paddle including a defibrillator electrode, the two paddles being electrically and physically connected to another by a flexible bridge and packaged together by a frangible seal; and
an electrical module incorporated within the portable defibrillator including electrical circuit, a battery and a capacitor, wherein the two paddles are sized and shaped as padded gloves or mittens.

6. The portable defibrillator as claimed in claim 5, further comprising one or more features selected from the group consisting of, wherein the electrical module is split between the two paddles and/or the bridge, wherein the electrical module is contained primarily within one paddle, wherein the bridge member includes indicia in the form of words and/or graphics to guide in the use of the portable defibrillator, wherein the electrical module includes a GPS module, wherein the electrical module includes a calling device, which calling device preferably comprises a wireless calling device, wherein the electrical module includes a self-test module, wherein the self-test module preferably monitors battery strength and/or electrode moisture, wherein the electrical module includes a memory and speakers for providing voice prompts for use of the defibrillator, and wherein the electrical module includes a detector for checking the victim's ECG rhythm.

7. A method of using a portable defibrillator comprising:
providing a portable defibrillator with a housing having a first housing piece and a second housing piece with a wire therebetween, wherein each of the first and second housing pieces has an electrode, wherein a battery and a capacitor are positioned within at least one of the first and second housing pieces, and wherein the housing has a spatial volume of less than about 26 inches (66 cm³) and linear three dimension sum of less than about 14 inches (35.5 cm); and
separating the first housing piece from the second housing piece to activate the portable defibrillator, wherein, after activation, the controller controls a release of a voltage from the battery to at least one of the electrodes of the first and second housing pieces.
